(19) United States
(12) Patent Application Publication
(10) Pub. No.: US 2017/0218005 A1
(43) Pub. Date: Aug. 3, 2017

(54) COMPOSITIONS AND METHODS RELATED TO GENTAMICIN

(71) Applicant: La Jolla Pharmaceutical Company, San Diego, CA (US)

(72) Inventors: George Tidmarsh, Portola Valley, CA (US); James Rolke, San Diego, CA (US); Lakhmir Chawla, San Diego, CA (US)

(21) Appl. No.: 15/417,698

(22) Filed: Jan. 27, 2017

Related U.S. Application Data
(60) Provisional application No. 62/288,866, filed on Jan. 29, 2016.

(51) Int. Cl. C07H 15/236; A61K 31/573; A61K 45/06; A61K 31/7036

(52) U.S. Cl. CPC C07H 15/236; A61K 31/7036; A61K 31/573; A61K 45/06

(57) ABSTRACT

Various aspects of the invention relate to compositions comprising C-2a gentamicin. A composition may be substantially free from one or more of C-1 gentamicin, C-1a gentamicin, C-2 gentamicin, C-2b gentamicin, gentamicin A, gentamicin B, and gentamicin X. In some aspects, the invention relates to a method for treating a condition in a subject, comprising administering a composition comprising C-2a gentamicin to the subject. The condition may be, for example, a bacterial infection, genetic disease or disorder, or cancer.

### RELATED APPLICATIONS

This application claims the benefit of priority to U.S. Provisional Patent Application Ser. No. 62/288,866, filed Jan. 29, 2016, which is incorporated herein by reference in its entirety.

### BACKGROUND

Gentamicin is the most widely used aminoglycoside antibiotic, with over 3 million doses administered annually in the United States. The therapeutic window for parenteral dosage forms of gentamicin is narrow, however, and administration may cause nephrotoxicity or neurotoxicity, including irreversible ototoxicity. Recently, it has been discovered that various congeners of gentamicin display different efficacies and toxicities. Specifically, the C-2 congener of gentamicin was identified as exhibiting low nephrotoxicity, suggesting that purified C-2 gentamicin may be preferable to “native gentamicin” (see, e.g., U.S. Pat. No. 8,951,978). Aminoglycoside antibiotics with broader therapeutic windows remain desirable.

### SUMMARY

In some aspects, the invention relates to a composition comprising C-2a gentamicin. The composition may be substantially free from one or more of C-1 gentamicin, C-1a gentamicin, and C-2 gentamicin. In some aspects, the invention relates to a method for treating a condition in a subject, comprising administering a composition comprising C-2a gentamicin to the subject. The condition may be a bacterial infection, genetic disease or disorder, or cancer.

### BRIEF DESCRIPTION OF THE DRAWINGS

FIG. 1 consists of four bar graphs, labeled A, B, C, and D. Each graph depicts different test frequencies (x-axis; 8 kHz, 16 kHz, and 32 kHz) and the volume of test frequency required to generate a response in a test subject (y-axis; sound level in decibels (sound pressure level, “dB SPL”)). Graph A depicts the auditory brainstem response (ABR) in rats dosed with vehicle control. Graph B depicts the ABR in rats dosed with 39.6 mg/kg, 59.4 mg/kg, or 79.2 mg/kg native gentamicin. Graph C depicts the ABR in rats dosed with 39.6 mg/kg, 59.4 mg/kg, or 79.2 mg/kg C-2 gentamicin. Graph D depicts the ABR in rats dosed with 39.6 mg/kg, 59.4 mg/kg, or 79.2 mg/kg C-2a gentamicin.

FIG. 2 is a bar graph depicting average serum creatinine levels for rats on experimental days 5, 9, and 25 after administering a vehicle control, native gentamicin, C-2a gentamicin, or C-2 gentamicin at concentrations of 39.6 mg/kg, 59.4 mg/kg, or 79.2 mg/kg.

### DETAILED DESCRIPTION

Various aspects of the invention relate to the finding that the C-2a congener of gentamicin displays less toxicity in vivo than native gentamicin or the C-2 congener of gentamicin. In particular, previous experiments involving various congeners of gentamicin suggested that C-2 gentamicin has a superior therapeutic window relative to other congeners, including C-2a gentamicin, which is a stereoisomer of C-2 gentamicin (see, e.g., U.S. Pat. No. 8,951,978). Surprisingly, the in vivo studies disclosed herein, infra, suggest that that C-2a gentamicin has a superior therapeutic window relative to both C-2 gentamicin and native gentamicin. Specifically, and in contrast with prior findings, C-2 gentamicin displays increased ototoxicity relative to native gentamicin, whereas C-2a gentamicin displayed no significant ototoxicity.

### I. Compositions

Native gentamicin is purified from the bacteria Micromonospora purpurea, and contains C-1, C-1a, C-2, C-2a, A, B, and X gentamicin. The different congeners of group C gentamicin, C-1, C-1a, C-2, and C-2a, may be purified from native gentamicin, for example, using reverse phase HPLC (see, e.g., U.S. Pat. No. 8,951,978 (describing purification using a C18 HPLC column), hereby incorporated by reference in its entirety). Native gentamicin comprises about 20-35% C-1 gentamicin, about 10-35% C-1a gentamicin, about 20-40% C-2 gentamicin, about 5-25% C-2a gentamicin, about 0-10% gentamicin A, about 0-10% gentamicin B, about 0-10% gentamicin X, and about 0-10% of other gentamicins (Vydrin, A. F. et al. PHARMACEUTICAL CHEM. J. 37(8):448 (2003)).

In some aspects, the invention relates to a composition comprising C-2a gentamicin. C-2a gentamicin has a structure of formula (I), wherein R₁ and R₂ are hydrogen and R₃ is methyl.

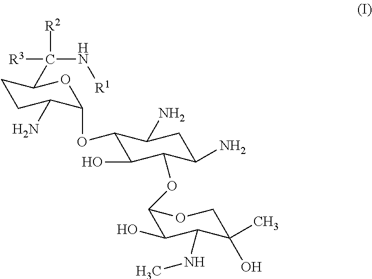

The composition may be substantially free from one or more of C-1 gentamicin, C-1a gentamicin, and C-2 gentamicin. C-1 gentamicin has the structure of formula (I), wherein R₁ and R₂ are methyl and R₃ is hydrogen. C-1a gentamicin has the structure of formula (I), wherein R₁, R₃, and R₂ are hydrogen. C-2 gentamicin has the structure of formula (I), wherein R₁ and R₃ are hydrogen and R₂ is methyl. The composition may be substantially free from C-2b gentamicin, which has the structure of formula (I), wherein R₂ and R₃ are hydrogen and R₁ is methyl. The composition may be substantially free from one or more of gentamicin A, gentamicin B, and gentamicin X.

The composition may be substantially free from two or three of C-1 gentamicin, C-1a gentamicin, and C-2 gentamicin. For example, the composition may comprise less than 5% C-1 gentamicin, less than 5% C-1a gentamicin, and/or less than 5% C-2 gentamicin. The composition may comprise less than 1% C-1 gentamicin, less than 1% C-1a gentamicin, and/or less than 1% C-2 gentamicin. The composition may comprise less than 1000 ppm of C-1 gentamicin, less than 1000 ppm of C-1a gentamicin, and/or less than 1000 ppm of C-2 gentamicin. The composition may comprise less than 100 ppm of C-1 gentamicin, less than 100 ppm of C-1a gentamicin, and/or less than 100 ppm of C-2 gentamicin. The composition may comprise less than 10 ppm of C-1 gentamicin, less than 10 ppm of C-1a gentamicin, and/or less than 10 ppm of C-2 gentamicin.

The composition may be substantially free from two or three of gentamicin A, gentamicin B, and gentamicin X. For example, the composition may comprise less than 5% gentamicin A, less than 5% gentamicin B, and/or less than 5% gentamicin X. The composition may comprise less than 1% gentamicin A, less than 1% gentamicin B, and/or less than 1% gentamicin X. The composition may comprise less than 1000 ppm of gentamicin A, less than 1000 ppm of gentamicin B, and/or less than 1000 ppm of gentamicin X. The composition may comprise less than 100 ppm of gentamicin A, less than 100 ppm of gentamicin B, and/or less than 100 ppm of gentamicin X. The composition may comprise less than 10 ppm of gentamicin A, less than 10 ppm of gentamicin B, and/or less than 10 ppm of gentamicin X.

The composition may be substantially free from C-2b gentamicin. For example, the composition may comprise less than 5% C-2b gentamicin. The composition may comprise less than 1% C-2b gentamicin. The composition may comprise less than 1000 ppm of C-2b gentamicin. The composition may comprise less than 100 ppm of C-2b gentamicin. The composition may comprise less than 10 ppm of C-2b gentamicin.

In some embodiments, the composition comprises less than 1000 ppm C-1 gentamicin, such as less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or even less than 10 ppm C-1 gentamicin.

In some preferred embodiments, the composition comprises less than 1000 ppm C-1a gentamicin, such as less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or even less than 10 ppm C-1a gentamicin.

In some embodiments, the composition comprises less than 1000 ppm C-2 gentamicin, such as less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or even less than 10 ppm C-2 gentamicin.

In some embodiments, the composition comprises less than 1000 ppm C-2b gentamicin, such as less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or even less than 10 ppm C-2b gentamicin.

In some embodiments, the composition comprises less than 1000 ppm gentamicin A, such as less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or even less than 10 ppm gentamicin A.

In some embodiments, the composition comprises less than 1000 ppm gentamicin B, such as less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or even less than 10 ppm gentamicin B.

In some embodiments, the composition comprises less than 1000 ppm gentamicin X, such as less than 900 ppm, less than 800 ppm, less than 700 ppm, less than 600 ppm, less than 500 ppm, less than 400 ppm, less than 300 ppm, less than 200 ppm, less than 100 ppm, less than 90 ppm, less than 80 ppm, less than 70 ppm, less than 60 ppm, less than 50 ppm, less than 40 ppm, less than 30 ppm, less than 20 ppm, or even less than 10 ppm gentamicin X.

The composition may have a ratio of C-1 gentamicin to C-2a gentamicin of less than about 1:10 (w/w); the composition may have a ratio of C-1a gentamicin to C-2a gentamicin of less than about 1:10 (w/w); and/or the composition may have a ratio of C-2 gentamicin to C-2a gentamicin of less than about 1:10 (w/w). The composition may have a ratio of C-1 gentamicin to C-2a gentamicin of less than about 1:20 (w/w); the composition may have a ratio of C-1a gentamicin to C-2a gentamicin of less than about 1:20 (w/w); and/or the composition may have a ratio of C-2 gentamicin to C-2a gentamicin of less than about 1:20 (w/w). The composition may have a ratio of C-1 gentamicin to C-2a gentamicin of less than about 1:50 (w/w); the composition may have a ratio of C-1a gentamicin to C-2a gentamicin of less than about 1:50 (w/w); and/or the composition may have a ratio of C-2 gentamicin to C-2a gentamicin of less than about 1:50 (w/w).

The composition may have a ratio of gentamicin A to C-2a gentamicin of less than about 1:10 (w/w); the composition may have a ratio of gentamicin B to C-2a gentamicin of less than about 1:10 (w/w); and/or the composition may have a ratio of gentamicin X to C-2a gentamicin of less than about 1:10 (w/w). The composition may have a ratio of gentamicin A to C-2a gentamicin of less than about 1:20 (w/w); the composition may have a ratio of gentamicin B to C-2a gentamicin of less than about 1:20 (w/w); and/or the composition may have a ratio of gentamicin X to C-2a gentamicin of less than about 1:20 (w/w). The composition may have a ratio of gentamicin A to C-2a gentamicin of less than about 1:50 (w/w); the composition may have a ratio of gentamicin B to C-2a gentamicin of less than about 1:50 (w/w); and/or the composition may have a ratio of gentamicin X to C-2a gentamicin of less than about 1:50 (w/w).

The composition may have a ratio of C-2b gentamicin to C-2a gentamicin of less than about 1:10 (w/w). The composition may have a ratio of C-2b gentamicin to C-2a gentamicin of less than about 1:20 (w/w). The composition may have a ratio of C-2b gentamicin to C-2a gentamicin of less than about 1:50 (w/w).

At least about 50% of the gentamicin in a composition may be C-2a gentamicin. At least about 60%, 65%, 70%, 75%, 80%, 85%, 90%, 95%, 96%, 97%, 98%, 99%, 99.5%, 99.8%, 99.9%, 99.95%, 99.98%, or 99.99% of the gentamicin in a composition may be C-2a gentamicin.

In preferred embodiments, a composition has a ratio of C-2a gentamicin to total gentamicin of greater than 9:10 (w/w), such as greater than greater than 19:20 (w/w), greater than 49:50 (w/w), greater than 99:100 (w/w), greater than 199:200 (w/w), or even greater than 499:500 (w/w).

The composition may comprise total gentamicin at a combined concentration of about 100 μg/mL to about 1 g/mL, such as about 0.5 mg/mL to about 200 mg/mL. The composition may comprise total gentamicin (e.g., C-1, C-1a, C-2, C-2a, C-2b, A, B, and X gentamicin) at a combined concentration of about 0.5 mg/mL to about 5 mg/mL, about 5 mg/mL to about 25 mg/mL, about 25 mg/mL to about 75 mg/mL, or about 75 mg/mL to about 125 mg/mL. The composition may comprise total gentamicin at a concentration of greater than about 100 μg/mL, such as greater than about 0.5 mg/mL, about 1 mg/mL, about 10 mg/mL, or greater than about 100 mg/mL. The composition may comprise total gentamicin (e.g., C-1, C-1a, C-2, C-2a, C-2b, A, B, and X gentamicin) at a combined concentration of about 0.8 mg/mL, about 1 mg/mL, about 1.2 mg/mL, about 1.4 mg/mL, about 1.6 mg/mL, about 1.8 mg/mL, about 2 mg/mL, about 2.4 mg/mL, about 3 mg/mL, about 6 mg/mL, about 9 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, or about 120 mg/mL.

The composition may comprise C-2a gentamicin at a concentration of about 100 μg/mL to about 1 g/mL, such as about 0.5 mg/mL to about 200 mg/mL. The composition may comprise C-2a gentamicin at a concentration of about 0.5 mg/mL to about 5 mg/mL, about 5 mg/mL to about 25 mg/mL, about 25 mg/mL to about 75 mg/mL, or about 75 mg/mL to about 125 mg/mL. The composition may comprise C-2a gentamicin at a concentration of greater than about 100 μg/mL, such as greater than about 0.5 mg/mL, about 1 mg/mL, about 10 mg/mL, or greater than about 100 mg/mL. The composition may comprise C-2a gentamicin at a concentration of about 0.8 mg/mL, about 1 mg/mL, about 1.2 mg/mL, about 1.4 mg/mL, about 1.6 mg/mL, about 1.8 mg/mL, about 2 mg/mL, about 2.4 mg/mL, about 3 mg/mL, about 6 mg/mL, about 9 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, or about 120 mg/mL.

In some embodiments, the composition is a liquid and the composition comprises gentamicin (e.g., C-1, C-1a, C-2, C-2a, C-2b, A, B, and X gentamicin) at a concentration about 0.1 mg/mL to about 115 mg/mL, such as about 0.3 mg/mL to about 30 mg/mL or about 10 mg/mL to about 100 mg/mL. The composition may comprise gentamicin at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL. In some preferred embodiments, the composition is a liquid, and the composition comprises gentamicin at a concentration of about 3 mg/mL, 10 mg/mL, or about 40 mg/mL.

The composition may comprise C-2a gentamicin at a concentration of about 1 mg/mL, about 2 mg/mL, about 3 mg/mL, about 4 mg/mL, about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL, about 15 mg/mL, about 16 mg/mL, about 17 mg/mL, about 18 mg/mL, about 19 mg/mL, about 20 mg/mL, about 21 mg/mL, about 22 mg/mL, about 23 mg/mL, about 24 mg/mL, about 25 mg/mL, about 26 mg/mL, about 27 mg/mL, about 28 mg/mL, about 29 mg/mL, about 30 mg/mL, about 31 mg/mL, about 32 mg/mL, about 33 mg/mL, about 34 mg/mL, about 35 mg/mL, about 36 mg/mL, about 37 mg/mL, about 38 mg/mL, about 39 mg/mL, about 40 mg/mL, about 41 mg/mL, about 42 mg/mL, about 43 mg/mL, about 44 mg/mL, about 45 mg/mL, about 46 mg/mL, about 47 mg/mL, about 48 mg/mL, about 49 mg/mL, or about 50 mg/mL. In some preferred embodiments, the composition is a liquid, and the composition comprises C-2a gentamicin at a concentration of about 3 mg/mL, 10 mg/mL, or about 40 mg/mL.

In some embodiments, the composition is a liquid, and the composition comprises gentamicin (e.g., C-1, C-1a, C-2, C-2a, C-2b, A, B, and X gentamicin) at a concentration about 0.01% to about 10%, such as about 0.05% to about 5%. The composition may comprise gentamicin at a concentration of about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, or about 5%. In some preferred embodiments, the composition is a liquid, and the composition comprises gentamicin at a concentration of about 0.3%.

In some embodiments, the composition is a liquid, and the composition comprises C-2a gentamicin at a concentration of about 0.01% to about 10%, such as about 0.05% to about 5%. The composition may comprise C-2a gentamicin at a concentration of about 0.05%, about 0.06%, about 0.07%, about 0.08%, about 0.09%, about 0.1%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1%, about 2%, about 3%, or about 5%. In some preferred embodiments, the composition is a liquid, and the composition comprises C-2a gentamicin at a concentration of about 0.3%.

Gentamicin may form a salt (e.g., with an acid), and thus gentamicin may be present in the composition as a salt. In some embodiments, the salt may be that of an inorganic acid, such as sulfate, hydrogen sulfate, phosphate, or chloride. In some embodiments, the salt may be that of an organic acid, such as a chiral salt, acetate, trifluoroacetate, carbonate, or tartrate. In preferred embodiments, the salt is a sulfate and/or hydrogen sulfate salt. The salt may be a tartrate, malate, and/or acetate salt.

The composition may comprise a preservative such as methylparaben, propylparaben, benzalkonium chloride, or sodium metabisulfite. The composition may comprise a chelating agent such as ethylenediaminetetraacetate. The composition may comprise sodium chloride. The composition may comprise water.

The composition may be a pharmaceutical composition. The composition may be a liquid, cream, or ointment. The composition is preferably sterile.

The composition may comprise a second active agent, such as a corticosteroid. The second active agent may be, for example, prednisolone.

### II. Packaging

In some aspects, the invention relates to a container comprising a composition as described herein. For example, the container may be a vial, e.g., for liquid compositions. The container may comprise glass, e.g., the container may be a glass vial.

In preferred embodiments, the vial is sealed. For example, the vial may be sealed with a stopper, or the vial may have a flip-top seal. The stopper may comprise, for example, rubber, metal, and/or plastic.

In preferred embodiments, the vial is sterile.

In preferred embodiments, the vial comprises at least enough of the composition to provide at least 1 mg of gentamicin. For example, the vial may comprise about 10 μl to about 100 mL of a liquid composition as described herein, such as about 400 μl and to 40 mL, such as about 500 μl, about 1 mL, about 2 mL, about 5 mL, about 10 ml, or about 20 mL.

The size of the vial is not particularly limiting. For example, the vial may have a volume of about 0.1 mL, 0.2 mL, 0.5 mL, 1 mL, 2 mL, 3 mL, 4 mL, 5 mL, 6 mL, 7 mL, 8 mL, 9 mL, or 10 mL. In preferred embodiments, the volume of the vial is about 0.1 mL to about 5 mL, such as 0.1 mL, 0.2 mL, 0.5 mL, 1 mL, 2 mL, or 3 mL.

### III. Administration of Gentamicin

In some aspects, the invention relates to a method for treating a condition in a subject, comprising administering a composition comprising C-2a gentamicin (e.g., as described herein) to the subject. Administering the composition may comprise intramuscular administration, intravenous administration, topical administration, administration to an eye, or administration to an ear. For example, the composition may be administered intravenously over the course of about 10 minutes to about 4 hours, such as over the course of about 30 minutes to about 180 minutes. Administering the composition may comprise contacting the composition with an ear or eye. For example, the condition may be bacterial conjunctivitis, and administration may comprise contacting the composition with an eye, e.g., the cornea and/or sclera of an eye. Administering a composition may comprise contacting the composition with an ear canal.

The condition may be a bacterial infection. A bacterial infection may be an infection caused by a species of Citrobacter, Enterobacter, Enterococcus, Escherichia, Francisella, Klebsiella, Listeria, Proteus, Pseudomonas, Serratia, Streptococcus, Staphylococcus, or Yersinia. The species may be, for example, Enterobacter aerogenes, Enterobacter cloacae, Enterococcus faecalis, Enterococcus faecium, Enterococcus durans, Escherichia coli, Francisella tularensis, Klebsiella pneumonia, Klebsiella oxytoca, Proteus mirabilis, Proteus penneri, Proteus vulgaris, Pseudomonas aeruginosa, Serratia liquefaciens, Serratia marcescens, Serratia odoriferae, Serratia plymuthica, Serratia rubidaea, Staphylococcus aureus, or Yersinia pestis. The bacterial infection may be bacterial conjunctivitis. In some embodiments, the identity of the bacteria may be unknown. The method may further comprise administering a second antibiotic to the subject. The second antibiotic may be a β-lactam, penicillin, carboxypenicillin, or cephalosporin. The antibiotic may be, for example, ampicillin, carbenicillin, nafcillin, oxacillin, or penicillin G. In some embodiments, the bacterial infection is life-threatening.

The condition may be a bone infection, endocarditis, pelvic inflammatory disease, meningitis, pneumonia, tularemia, a urinary tract infection, or sepsis. The condition may be a burn.

In some aspects, the invention relates to a method for preventing a condition in a subject, comprising administering a composition comprising C-2a gentamicin (e.g., as described herein) to the subject. The term “preventing” is art-recognized, and when used in relation to a condition such as a local occurrence (e.g., bacterial infection), a disease such as cellulitis, erysipelas, or impetigo, or an injury such as scarring, is well understood in the art, and includes administration of a composition which reduces the frequency of, or delays the onset of, symptoms of a medical condition in a subject relative to a subject which does not receive the composition. Thus, prevention of a bacterial infection includes, for example, reducing the number of infections in a population of patients receiving a prophylactic treatment relative to an untreated control population, and/or delaying the appearance of infections in a treated population versus an untreated control population, e.g., by a statistically and/or clinically significant amount. For example, a method may comprise administering gentamicin (e.g., C-2a gentamicin) to a subject who has a burn to prevent a bacterial infection secondary to the burn, e.g., wherein administering gentamicin comprises topical administration.

The condition may be a genetic disease or disorder. The genetic disease or disorder may be caused by a premature stop codon in a gene, e.g., the genetic disease or disorder may be caused by a nonsense mutation. The genetic disease or disorder may be cystic fibrosis, Hurler syndrome, Scheie syndrome, Duchenne muscular dystrophy, β-thalassemia, McArdle's disease, or Becker muscular dystrophy.

The condition may be cancer. The cancer may comprise a premature stop codon in a gene, e.g., a neoplastic cell or metastatic cell may comprise a premature stop codon in a gene, such as in a gene encoding p53.

The method may comprise administering greater than about 100 μg/kg body weight total gentamicin to the subject, such as greater than about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or greater than about 10 mg/kg total gentamicin to the subject.

The method may comprise administering greater than about 100 μg/kg body weight C-2a gentamicin to the subject, such as greater than about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or greater than about 10 mg/kg C-2a gentamicin to the subject.

The method may comprise administering about 100 μg/kg body weight to about 20 mg/kg body weight total gentamicin to the subject, such as about 500 μg/kg body weight to about 10 mg/kg body weight total gentamicin to the subject. The method may comprise administering about 100 μg/kg body weight to about 20 mg/kg body weight C-2a gentamicin to the subject, such as about 500 μg/kg body weight to about 10 mg/kg body weight C-2a gentamicin to the subject.

The method may comprise administering about 100 μg/kg body weight total gentamicin to the subject. The method may comprise administering about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or about 10 mg/kg total gentamicin to the subject.

The method may comprise administering about 100 μg/kg body weight C-2a gentamicin to the subject. The method may comprise administering about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or about 10 mg/kg C-2a gentamicin to the subject.

The method may comprise administering the composition to the subject once, twice, three times, or more. The method may comprise administering the composition to the subject 1 time per day, 2 times per day, 3 times per day, 4 times per day, or 5 times per day. For example, the method may comprise administering the composition to the subject once every 6, 8, 12, or 24 hours. The method may comprise re-administering the composition to the subject. The method may comprise re-administering the composition to the subject once every 6, 8, 12, or 24 hours, e.g., for a period of time selected from 5 days to 20 days, such as from 7 to 10 days.

The method may comprise administering greater than about 100 μg/kg body weight total gentamicin to the subject per day, such as greater than about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or greater than about 10 mg/kg total gentamicin to the subject per day.

The method may comprise administering greater than about 100 μg/kg body weight C-2a gentamicin to the subject per day, such as greater than about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or greater than about 10 mg/kg C-2a gentamicin to the subject per day.

The method may comprise administering about 100 μg/kg body weight to about 20 mg/kg body weight total gentamicin to the subject per day, such as about 500 μg/kg body weight to about 10 mg/kg body weight total gentamicin to the subject per day. The method may comprise administering about 100 μg/kg body weight to about 20 mg/kg body weight C-2a gentamicin to the subject per day, such as about 500 μg/kg body weight to about 10 mg/kg body weight C-2a gentamicin to the subject per day.

The method may comprise administering about 100 μg/kg body weight total gentamicin to the subject per day. The method may comprise administering about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or about 10 mg/kg total gentamicin to the subject per day.

The method may comprise administering about 100 μg/kg body weight C-2a gentamicin to the subject per day. The method may comprise administering about 150 μg/kg, about 200 μg/kg, 250 μg/kg, about 300 μg/kg, 350 μg/kg, about 400 μg/kg, 450 μg/kg, about 500 μg/kg, 550 μg/kg, about 600 μg/kg, 650 μg/kg, about 700 μg/kg, 750 μg/kg, about 800 μg/kg, 850 μg/kg, about 900 μg/kg, 950 μg/kg, about 1000 μg/kg, about 1.5 mg/kg, about 1.7 mg/kg, about 2.0 mg/kg, 2.5 mg/kg, about 3.0 mg/kg, 3.5 mg/kg, about 4.0 mg/kg, 4.5 mg/kg, about 5.0 mg/kg, 5.5 mg/kg, about 6.0 mg/kg, 6.5 mg/kg, about 7.0 mg/kg, 7.5 mg/kg, about 8.0 mg/kg, 8.5 mg/kg, about 9.0 mg/kg, 9.5 mg/kg, or about 10 mg/kg C-2a gentamicin to the subject per day.

Typically, if the composition is administered to treat a bacterial infection, the composition is administered at about 1 mg/kg body weight total gentamicin (or C-2a gentamicin) to about 10 mg/kg body weight for about 5 to about 14 days, such as about 7 to 10 days.

### IV. Subjects

A subject may be a rodent, lagomorph, feline, canine, porcine, ovine, bovine, equine, or primate. In preferred embodiments, the subject is a human.

A subject may be an adult, child, infant, neonate, premature neonate, or full-term neonate. An elevated dose of gentamicin (e.g., C-2a gentamicin) may be administered to a child, infant, or neonate relative to an adult. For example, a child, infant, or neonate may receive 1.5×, 2.0×, or 2.5× gentamicin (e.g., C-2a gentamicin) relative to an adult.

A subject may have a bacterial infection. A subject may have a genetic disease or disorder. The subject may have cancer. In some embodiments, a subject has a life-threatening infection, i.e., a life-threatening bacterial infection.

A subject may have a bone infection, endocarditis, pelvic inflammatory disease, meningitis, pneumonia, tularemia, a urinary tract infection, or sepsis. A subject may have a burn. A subject may have cystic fibrosis, Hurler syndrome, Scheie syndrome, Duchenne muscular dystrophy, β-thalassemia, McArdle's disease, or Becker muscular dystrophy.

A subject may have renal impairment. A subject may have a creatinine clearance rate less than 70 mL/min/1.73 m². A subject may have a creatinine clearance rate of about 70 mL/min/1.73 m² to about 100 mL/min/1.73 m². A subject may have a creatinine clearance rate greater than or equal to 100 mL/min/1.73 m².

A subject may have a serum creatinine concentration of greater than 1 mg/dL, such as greater than 1.1 mg/dL, greater than 1.4 mg/dL, greater than 1.7 mg/dL, greater than 2 mg/dL, greater than 2.3 mg/dL, greater than 2.6 mg/dL, greater than 3.1 mg/dL, greater than 3.6 mg/dL, greater than 4.1 mg/dL, greater than 5.2 mg/dL, or greater than 6.7 mg/dL. A subject may have a serum creatinine concentration of about 1.1 mg/dL to about 1.3 mg/dL, about 1.4 mg/dL to about 1.6 mg/dL, about 1.7 mg/dL to about 1.9 mg/dL, about 2.0 mg/dL to about 2.2 mg/dL, about 2.3 mg/dL to about 2.5 mg/dL, about 2.6 mg/dL to about 3.0 mg/dL, about 3.1 mg/dL to about 3.5 mg/dL, about 3.6 mg/dL to about 4.0 mg/dL, about 4.1 mg/dL to about 5.1 mg/dL, about 5.2 mg/dL to about 6.6 mg/dL, or about 6.7 mg/dL to about 8.0 mg/dL.

A subject may have a creatinine clearance rate of less than about 100 mL/min/1.73 m², less than about 90 mL/min/1.73 m², less than about 80 mL/min/1.73 m², less than about 70 mL/min/1.73 m², less than about 60 mL/min/1.73 m², less than about 50 mL/min/1.73 m², less than about 40 mL/min/1.73 m², less than about 30 mL/min/1.73 m², less than about 20 mL/min/1.73 m², or less than about 10 mL/min/1.73 m². A subject may have a creatinine clearance rate of about 10 mL/min/1.73 m² to about 15 mL/min/1.73 m², about 10 mL/min/1.73 m² to about 15 mL/min/1.73 m², about 15 mL/min/1.73 m² to about 20 mL/min/1.73 m², about 20 mL/min/1.73 m² to about 25 mL/min/1.73 m², about 25 mL/min/1.73 m² to about 30 mL/min/1.73 m², about 30 mL/min/1.73 m² to about 35 mL/min/1.73 m², about 35 mL/min/1.73 m² to about 40 mL/min/1.73 m², about 40 mL/min/1.73 m² to about 45 mL/min/1.73 m², about 45 mL/min/1.73 m² to about 55 mL/min/1.73 m², about 55 mL/min/1.73 m² to about 70 mL/min/1.73 m², or about 70 mL/min/1.73 m² to about 100 mL/min/1.73 m².

In some embodiments, the subject does not present with renal impairment. A subject may have a serum creatinine concentration of about 1 mg/dL or less. A subject may have a creatinine clearance rate of greater than about 100 mL/min/1.73 m².

The subject may have renal impairment. For example, a subject may have renal impairment, and the method may comprise administering greater than 0.1 mg/kg body weight gentamicin (e.g., C-2a gentamicin) to the subject, such as about 0.1 mg/kg to about 5 mg/kg body weight gentamicin (e.g., C-2a gentamicin). The method may comprise administering about 0.1 mg/kg body weight gentamicin (e.g., C-2a gentamicin) to the subject. The method may comprise administering about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.5 mg/kg, about 1.66 mg/kg, about 2 mg/kg, or about 2.5 mg/kg gentamicin (e.g., C-2a gentamicin) to the subject.

The subject may have an approximate creatinine clearance rate of less than 10 mL/min/1.73 m², and the method comprises administering about 0.1 mg/kg body weight to about 1 mg/kg body weight gentamicin (e.g., C-2a gentamicin) to the subject, such as about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, or about 1 mg/kg gentamicin (e.g., C-2a gentamicin) to the subject.

A subject may have an approximate creatinine clearance rate of about 5 mL/min/1.73 m² to about 10 mL/min/1.73 m², about 10 mL/min/1.73 m² to about 15 mL/min/1.73 m², about 15 mL/min/1.73 m² to about 20 mL/min/1.73 m², about 20 mL/min/1.73 m² to about 25 mL/min/1.73 m², about 25 mL/min/1.73 m² to about 30 mL/min/1.73 m², about 30 mL/min/1.73 m² to about 35 mL/min/1.73 m², about 35 mL/min/1.73 m² to about 40 mL/min/1.73 m², about 40 mL/min/1.73 m² to about 45 mL/min/1.73 m², about 45 mL/min/1.73 m² to about 55 mL/min/1.73 m², about 55 mL/min/1.73 m² to about 70 mL/min/1.73 m², or about 70 mL/min/1.73 m² to about 100 mL/min/1.73 m², and the method may comprise administering about 0.1 mg/kg body weight gentamicin to about 2.5 mg/kg body weight gentamicin (e.g., C-2a gentamicin) to the subject, such as about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.5 mg/kg, about 1.66 mg/kg, about 2 mg/kg, or about 2.5 mg/kg gentamicin (e.g., C-2a gentamicin) to the subject.

A subject may have an approximate creatinine clearance rate of greater than 1 mg/dL, such as greater than 1.1 mg/dL, greater than 1.4 mg/dL, greater than 1.7 mg/dL, greater than 2 mg/dL, greater than 2.3 mg/dL, greater than 2.6 mg/dL, greater than 3.1 mg/dL, greater than 3.6 mg/dL, greater than 4.1 mg/dL, greater than 5.2 mg/dL, or greater than 6.7 mg/dL, and the method may comprise administering about 0.1 mg/kg body weight to about 2.5 mg/kg body weight gentamicin (e.g., C-2a gentamicin) to the subject, such as about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.5 mg/kg, about 1.66 mg/kg, about 2 mg/kg, or about 2.5 mg/kg gentamicin (e.g., C-2a gentamicin) to the subject.

A subject may have a serum creatinine concentration of about 1.1 mg/dL to about 1.3 mg/dL, about 1.4 mg/dL to about 1.6 mg/dL, about 1.7 mg/dL to about 1.9 mg/dL, about 2.0 mg/dL to about 2.2 mg/dL, about 2.3 mg/dL to about 2.5 mg/dL, about 2.6 mg/dL to about 3.0 mg/dL, about 3.1 mg/dL to about 3.5 mg/dL, about 3.6 mg/dL to about 4.0 mg/dL, about 4.1 mg/dL to about 5.1 mg/dL, about 5.2 mg/dL to about 6.6 mg/dL, or about 6.7 mg/dL to about 8.0 mg/dL, and the method may comprise administering about 0.1 mg/kg body weight to about 2.5 mg/kg body weight gentamicin (e.g., C-2a gentamicin) to the subject, such as about 0.1 mg/kg, about 0.15 mg/kg, about 0.2 mg/kg, about 0.25 mg/kg, about 0.3 mg/kg, about 0.35 mg/kg, about 0.4 mg/kg, about 0.45 mg/kg, about 0.5 mg/kg, about 0.55 mg/kg, about 0.6 mg/kg, about 0.65 mg/kg, about 0.7 mg/kg, about 0.75 mg/kg, about 0.8 mg/kg, about 0.85 mg/kg, about 0.9 mg/kg, about 0.95 mg/kg, about 1 mg/kg, about 1.5 mg/kg, about 1.66 mg/kg, about 2 mg/kg, or about 2.5 mg/kg gentamicin (e.g., C-2a gentamicin) to the subject.

The invention will be more readily understood by reference to the following examples, which are included merely to illustrate certain aspects and embodiments of the present invention and are not intended to limit the invention.

### EXEMPLIFICATION

### Example 1: Auditory Brain Response (ABR) Thresholds Increased as Native Gentamicin Doses Increased

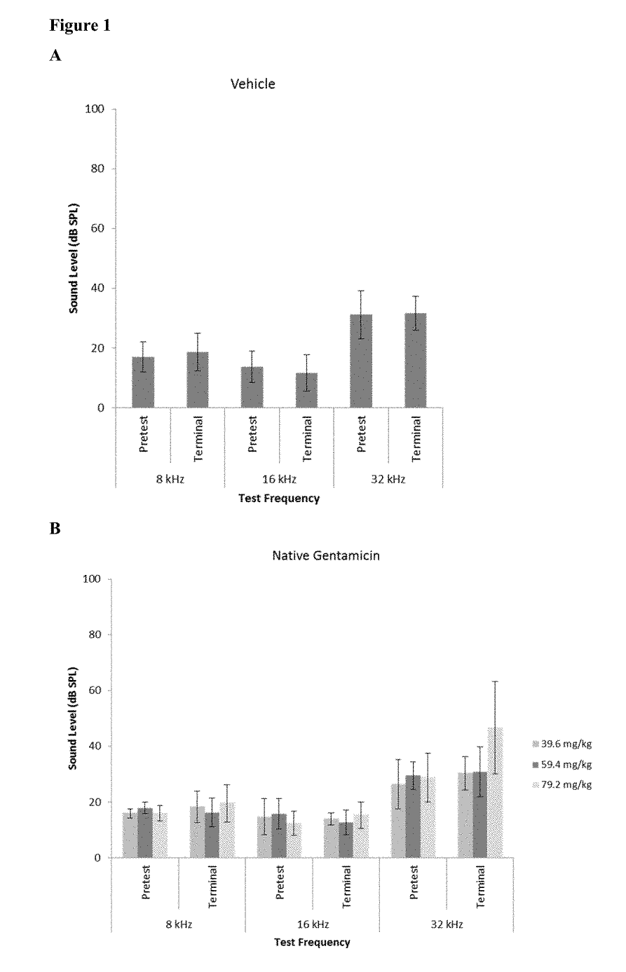

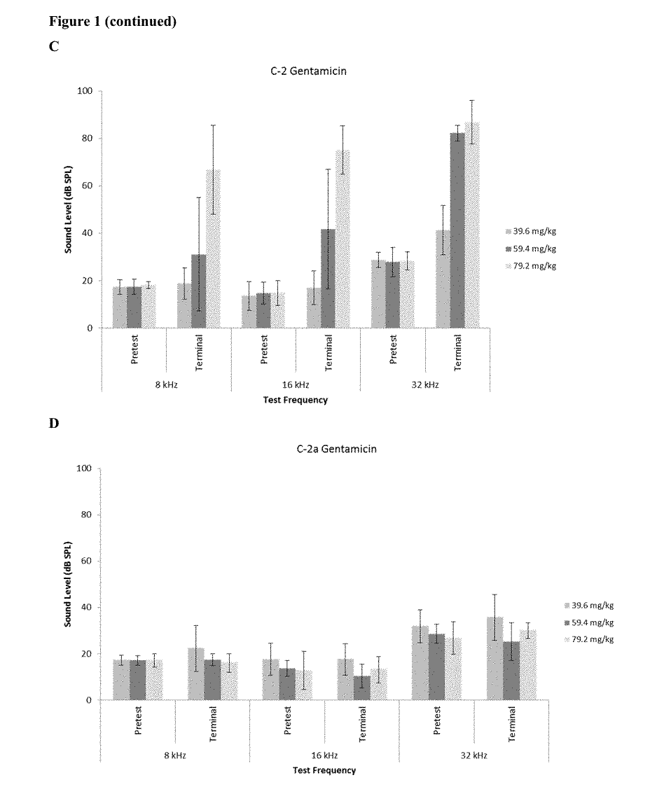

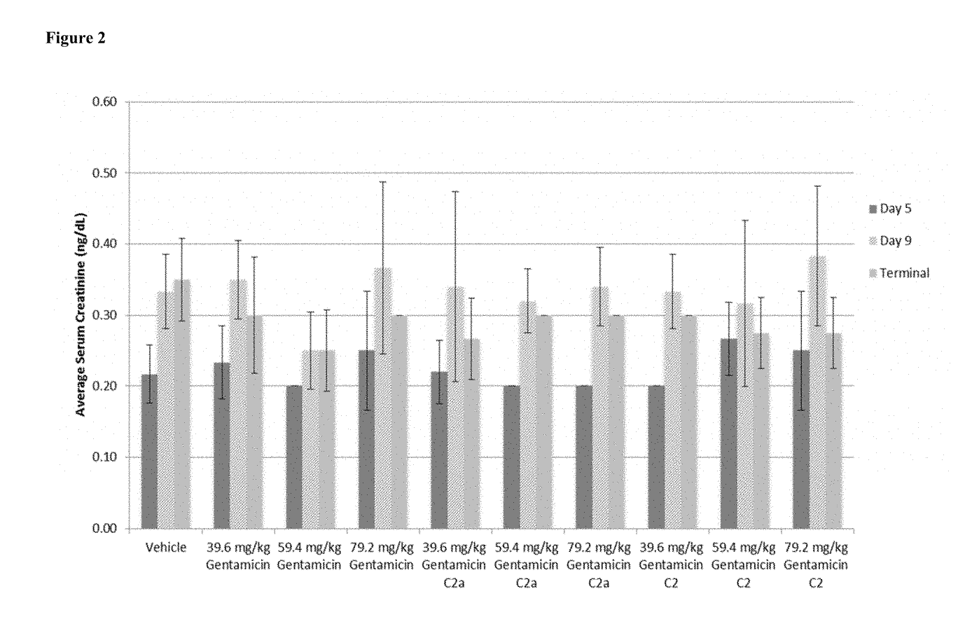

Male rats underwent nine intraperitoneal injections of native gentamicin (including C-1, C-1a, C-2, and C-2a gentamicin) at regular intervals over a nine day experimental period. Three experimental groups were dosed at concentrations of 39.6 mg/kg, 59.4 mg/kg, or 79.2 mg/kg native gentamicin. In addition, a vehicle control group underwent intraperitoneal injections of 0.9% NaCl vehicle. Auditory brainstem response (ABR) evaluations were conducted on all animals prior to the initial injection as well as on day 25, at test frequencies of 8 kHz, 16 KHz, and 32 KHz. ABR threshold, measured in sound pressure (decibels), increased as the native gentamicin dose increased. These results suggest native gentamicin causes hearing loss (FIGS. 1A and 1B).

### Example 2: Auditory Brainstem Response (ABR) Thresholds Increased Significantly as C-2 Gentamicin Doses Increased but ABR Thresholds Displayed No Increase with C-2a Gentamicin

Male rats received nine intraperitoneal injections of C-2a gentamicin or C-2 gentamicin at regular intervals over a nine days. Experimental groups were dosed with 39.6 mg/kg, 59.4 mg/kg, or 79.2 mg/kg of either C-2a gentamicin or C-2 gentamicin. The control group underwent intraperitoneal injections with a 0.9% NaCl vehicle (FIG. 1A). ABR evaluations were conducted on all animals prior to the initial injection as well as on day 25, at test frequencies of 8 kHz, 16 KHz, and 32 KHz. ABR thresholds did not significantly increase in rats injected with C-2a gentamicin at any dose relative to pre-test ABR thresholds, suggesting that C-2a gentamicin does not cause hearing loss (FIG. 1D). ABR thresholds increased significantly for C-2 gentamicin relative to ABR thresholds measured prior to injection, suggesting that C-2 gentamicin can cause hearing loss (FIG. 1C).

### Example 3: Native Gentamicin, C-2a Gentamicin, or C-2 Gentamicin Did not Significantly Alter Serum Creatinine Levels

Serum creatinine is an indicator of renal health, and elevated levels of serum creatinine correlate with impaired kidney function. In order to test the effect of native gentamicin, C-2a gentamicin, and C-2 gentamicin on renal function, serum creatinine levels were measured in the rats described in Examples 1 and 2. Specifically, serum creatinine levels were measured at experimental days 5, 9, and 25. Dosing with native gentamicin, C-2a gentamicin, and C-2 gentamicin did not significantly affect serum creatinine levels relative to rats dosed with the saline control, indicating that the doses of native gentamicin, C-2a gentamicin, and C-2 gentamicin did not significantly affect kidney function.

### INCORPORATION BY REFERENCE

All publications and patents mentioned herein are hereby incorporated by reference in their entirety as if each individual publication or patent was specifically and individually indicated to be incorporated by reference. In case of conflict, the present specification, including its specific definitions, will control. While specific aspects of the subject matter have been discussed, the above specification is illustrative and not restrictive. Many variations will become apparent to those skilled in the art upon review of this specification and the claims below. The full scope of the invention should be determined by reference to the claims, along with their full scope of equivalents, and the specification, along with such variations.

1. A composition comprising C-2a gentamicin, wherein:
the C-2a gentamicin has the structure of formula (I), wherein R₁ and R₂ are hydrogen and R₃ is methyl; 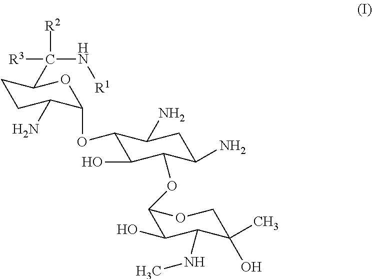
the composition is substantially free of one or more of C-1 gentamicin, C-1a gentamicin, and C-2 gentamicin; and
C-1 gentamicin has the structure of formula (I), wherein R₁ and R₂ are methyl and R₃ is hydrogen; C-1a gentamicin has the structure of formula (I), wherein R₁, R₃, and R₂ are hydrogen; and C-2 gentamicin has the structure of formula (I), wherein R₁ and R₃ are hydrogen and R₂ is methyl.

2. The composition of claim 1, wherein
the composition is substantially free of C-1a gentamicin.

3. The composition of claim 1, wherein
the composition is substantially free of C-2 gentamicin.

4. The composition of claim 1, wherein the composition comprises less than 5% C-1 gentamicin, less than 5% C-1a gentamicin, less than 5% C-2 gentamicin, less than 5% C-2b gentamicin, less than 5% gentamicin A, less than 5% gentamicin B, and/or less than 5% gentamicin X.

5. The composition of claim 1, wherein the composition is substantially free of two, three, four, five, six, or all of C-1 gentamicin, C-1a gentamicin, C-2 gentamicin, C-2b gentamicin, gentamicin A, gentamicin B, and gentamicin X.

6. The composition of claim 1, wherein the composition has a ratio of C-1 gentamicin to C-2a gentamicin of less than about 1:10 (w/w); the composition has a ratio of C-1a gentamicin to C-2a gentamicin of less than about 1:10 (w/w); the composition has a ratio of C-2 gentamicin to C-2a gentamicin of less than about 1:10 (w/w); the composition has a ratio of C-2b gentamicin to C-2a gentamicin of less than about 1:10 (w/w); the composition has a ratio of gentamicin A to C-2a gentamicin of less than about 1:10 (w/w); the composition has a ratio of gentamicin B to C-2a gentamicin of less than about 1:10 (w/w); and/or the composition has a ratio of gentamicin X to C-2a gentamicin of less than about 1:10 (w/w).

7. The composition of claim 1, wherein at least about 90% of the gentamicin in the composition is C-2a gentamicin.

8. The composition of claim 1, wherein the composition comprises total gentamicin at a combined concentration of about 0.5 mg/mL to about 200 mg/mL.

9. The composition of claim 8, wherein the composition comprises C-2a gentamicin at a concentration of about 0.5 mg/mL to about 200 mg/mL.

10. The composition of claim 8, wherein the composition comprises total gentamicin at a combined concentration of about 0.5 mg/mL to about 5 mg/mL, about 5 mg/mL to about 25 mg/mL, about 25 mg/mL to about 75 mg/mL, or about 75 mg/mL to about 125 mg/mL.

11. The composition of claim 10, wherein the composition comprises C-2a gentamicin at a concentration of about 0.5 mg/mL to about 5 mg/mL, about 5 mg/mL to about 25 mg/mL, about 25 mg/mL to about 75 mg/mL, or about 75 mg/mL to about 125 mg/mL.

12. The composition of claim 8, wherein the composition comprises total gentamicin at a combined concentration of about 0.8 mg/mL, about 1 mg/mL, about 1.2 mg/mL, about 1.4 mg/mL, about 1.6 mg/mL, about 1.8 mg/mL, about 2 mg/mL, about 2.4 mg/mL, about 3 mg/mL, about 6 mg/mL, about 9 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, or about 120 mg/mL.

13. The composition of claim 12, wherein the composition comprises C-2a gentamicin at a concentration of about 0.8 mg/mL, about 1 mg/mL, about 1.2 mg/mL, about 1.4 mg/mL, about 1.6 mg/mL, about 1.8 mg/mL, about 2 mg/mL, about 2.4 mg/mL, about 3 mg/mL, about 6 mg/mL, about 9 mg/mL, about 10 mg/mL, about 20 mg/mL, about 30 mg/mL, about 40 mg/mL, about 50 mg/mL, about 60 mg/mL, about 70 mg/mL, about 80 mg/mL, about 90 mg/mL, about 100 mg/mL, or about 120 mg/mL.

14. The composition of claim 1, wherein the gentamicin is present as a sulfate salt.

15. The composition of claim 1, wherein the composition is a liquid, cream, or ointment.

16. The composition of claim 1, wherein the composition further comprises a second active agent.

17. The composition of claim 16, wherein the second active agent is a corticosteroid.

18. The composition of claim 16, wherein the second active agent is prednisolone.

19. A method for treating or preventing a condition in a subject, comprising administering the composition of claim 1 to the subject.

20. The method of claim 19, wherein administering the composition comprises intramuscular administration, intravenous administration, topical administration, administration to an eye, or administration to an ear.

21. The method of claim 19, wherein the condition is a bacterial infection.

22. The method of claim 21, wherein the bacteria is a species of Citrobacter, Enterobacter, Enterococcus, Escherichia, Francisella, Klebsiella, Listeria, Proteus, Pseudomonas, Serratia, Streptococcus, Staphylococcus, or Yersinia.

23. The method of claim 22, wherein the bacteria is Enterobacter aerogenes, Enterobacter cloacae, Enterococcus faecalis, Enterococcus faecium, Enterococcus durans, Escherichia coli, Francisella tularensis, Klebsiella pneumonia, Klebsiella oxytoca, Proteus mirabilis, Proteus penneri, Proteus vulgaris, Pseudomonas aeruginosa, Serratia liquefaciens, Serratia marcescens, Serratia odoriferae, Serratia plymuthica, Serratia rubidaea, Staphylococcus aureus, or Yersinia pestis.

24. The method of claim 21, wherein the identity of the bacteria is unknown.

25. The method of claim 19, further comprising administering ampicillin, carbenicillin, a cephalosporin, nafcillin, oxacillin, or penicillin to the subject.

26. The method of claim 19, wherein the condition is a genetic disease or disorder.

27. The method of claim 26, wherein the genetic disease or disorder is caused by a premature stop codon in a gene.

28. The method of claim 26, wherein the genetic disease or disorder is cystic fibrosis, Hurler syndrome, Scheie syndrome, Duchenne muscular dystrophy, β-thalassemia, McArdle's disease, or Becker muscular dystrophy.

29. The method of claim 19, wherein the condition is cancer.

30. The method of claim 29, wherein the cancer comprises a nonsense mutation associated with a premature stop codon.

31. The method of claim 30, wherein the nonsense mutation is a mutation to the gene encoding p53.

32. The method of claim 19, comprising administering greater than about 2 mg/kg total gentamicin to the subject.

33. The method of claim 19, comprising administering about 0.5 mg/kg to about 20 mg/kg total gentamicin to the subject.

34. The method of claim 33, comprising administering about 1 mg/kg, about 1.7 mg/kg, about 2 mg/kg, about 2.5 mg/kg, about 3 mg/kg, about 5 mg/kg, about 6 mg/kg, or about 7.5 mg/kg total gentamicin to the subject.

35. The method of claim 32, comprising administering greater than about 2 mg/kg C-2a gentamicin to the subject per day.

36. The method of claim 33, comprising administering about 0.5 mg/kg to about 20 mg/kg C-2a gentamicin to the subject per day.

37. The method of claim 34, comprising administering about 1 mg/kg, about 1.7 mg/kg, about 2 mg/kg, about 2.5 mg/kg, about 3 mg/kg, about 5 mg/kg, about 6 mg/kg, or about 7.5 mg/kg C-2a gentamicin to the subject per day.

38. The method of claim 19, wherein the subject is an adult, child, infant, neonate, premature neonate, or full-term neonate.

39. The method of claim 19, wherein the subject is a rodent, lagomorph, feline, canine, porcine, ovine, bovine, equine, or primate.

40. The method of claim 39, wherein the subject is a human.

41. The method of claim 19, wherein the subject has renal impairment.

42. The method of claim 41, wherein the subject has a creatinine clearance rate less than 70 mL/min/1.73 m².

43. The method of claim 41, wherein the subject has a creatinine clearance rate of about 70 mL/min/1.73 m² to about 100 mL/min/1.73 m².

44. The method of claim 41, wherein the subject has a creatinine clearance rate greater than or equal to 100 mL/min/1.73 m².

45. The composition of claim 2, wherein the composition is substantially free of C-2 gentamicin.
